(19) United States
(12) Patent Application Publication
(10) Pub. No.: US 2020/0330366 A1
(43) Pub. Date: Oct. 22, 2020

(54) COSMETIC USE OF A PROTEIN EXTRACT OF MORINGA OLEIFERA SEEDS

(71) Applicant: BASF BEAUTY CARE SOLUTIONS FRANCE SAS, Lyon (FR)

(72) Inventors: Louis DANOUX, Saulxures-Lès-Nancy (FR); Pierre DEPOUILLY, Paris (FR); Sabrina LEOTY-OKOMBI, Vaulx Milieu (FR); Boris VOGELGESANG, Lyon (FR)

(21) Appl. No.: 16/960,638

(22) PCT Filed: Jan. 9, 2019
(86) PCT No.: PCT/FR2019/050036

§ 371 Date: Jul. 8, 2020

(30) Foreign Application Priority Data
Jan. 9, 2018 (FR) 1850156

(51) Int. Cl. A61K 8/9789; A61Q 19/00

(52) U.S. Cl. CPC A61K 8/9789; A61Q 19/005

(57) ABSTRACT

The invention relates to the cosmetic use of a protein extract of defatted non-germinated Moringa oleifera seeds for the treatment of sensitive, sensitized, reactive, fragile and/or weakened skin and/or mucous membranes. The invention also relates to a protein extract of defatted non-germinated Moringa oleifera seeds for use in the treatment and/or prevention and/or reduction of the occurrence of pathologies related to sensitive, sensitized, reactive, fragile, weakened, intolerant, hyperreactive and/or irritated skin and/or mucous membranes, such as contact urticaria, irritative or allergic contact dermatitis, eczema, psoriasis, seborrheic or atopic dermatitis, and/or in the treatment and/or prevention of inflammation and/or irritation, in particular caused by Staphylococcus aureus, and/or in the treatment and/or prevention of erythema, in particular diaper rash.

The present invention concerns the field of cosmetology and dermatology, more particularly cosmetics for care of skin and mucosa, in particular sensitive, sensitized, reactive, fragile and/or weakened skin and/or mucosa and the use of an ungerminated, deoiled Moringa seed protein extract.

The Moringa genus includes some 14 plant species (including Moringa peregrina, M. aptera, M. concanensis, M. drouhardii, M. hildebrandtii, M. longituba), among which Moringa pterygosperma (also called Moringa oleifera) is the best known.

In this case, it is a quickly-growing tree that adapts very well to variable conditions, growing throughout the tropics, in Asia, Africa and South America. Fruits of 30 to 50 cm long, hang like drum sticks, hence the English name “drumstick tree”, and its green pods are appreciated as a vegetable all over the world. As a result, seeds are rarely left to ripen for oil production.

The different parts of the tree (leaves, roots, root bark, flowers and seeds) are used in traditional medicine in the countries where they grow.

Moringa seeds are characterized by the presence of an oil whose content varies from 21 to 53% depending on the species and maturity of the seeds. For the Moringa oleifera species, the contents mentioned in the literature range from 21 to 34%.

Due to its excellent stability with regard to oxidation and its good properties of fixing fragrances, Moringa oil, also called Behen or Ben oil, was the oil most used by ointment formulators for cosmetic and religious uses in ancient civilizations. This oil was used by cosmetic formulators up to the last century and its use has been recently “rediscovered”.

In addition to their oil content, Moringa seeds have more recently drawn the attention of researchers for their protein extract. Thus, application EP1064008 describes the use of a protein extract of Moringa seeds on the skin and the mucosa for its effects of softening, physiological conditioning, moisturizing, restructuring, repairing, and as an antiwrinkle and antipollution agent.

In particular, such an extract is sold by BASF under the name Purisoft® for its purifying and antipollution effect.

The article by ARMAND-STUSSI et al. (Personal Care, May 2003) also describes the purifying and antipollution effect (by combatting the harmful effects of pollution, such as cigarette smoke or heavy metals, on the skin and by eliminating or facilitating the elimination of microparticles on the skin such as coal dust) of the extract sold by BASF under the name Purisoft®.

Application WO02/096448 describes the use of a protein extract of delipidated or non-delipidated seed in the fields of deodorizing, removing unpleasant odors, cleaning, intimate hygiene, oral hygiene and dental care. In particular, this document also describes that such an extract has a soothing, softening and moisturizing effect and reduces feelings of fatigue. However, this soothing effect has at no time been suggested to be linked to a reduction of skin reactivity or treatment of skin and/or mucosa inflammation. Moreover, this document indicates that the extract maintains the balance of bacterial and fungal flora on mucosal surfaces, but only in the sense that it does not damage them, unlike other conventional deodorants. Thus, this application neither describes nor suggests that the extract can be used in the treatment of sensitive, sensitized, reactive, fragile, weakened and/or intolerant skin and/or mucosa, in particular by reducing inflammation, nor that it can have a beneficial effect on the beneficial commensal flora of the skin and/or mucosa, by preserving and/or increasing it.

Application FR2946879 also describes a Moringa extract for its cosmetic use. However, it is a question of a whole, i.e., not deoiled, seed extract.

Application CN103223000 further describes the use of a germinated Moringa seed extract in cosmetics, in particular obtained by extraction using a supercritical fluid, an extraction method that extracts the oily compounds.

The present invention therefore concerns a novel use of a protein extract of ungerminated, deoiled Moringa oleifera seed that has never been described or suggested in the prior art.

Indeed, the inventors have discovered that such an extract permits inhibiting the release of proinflammatory cytokines IL-6 and IL-8, in particular induced by the opportunistic pathogenic bacteria S. aureus (Example 2). This extract therefore has an effect on the skin inflammation processes by reducing them, and therefore is a product of choice in the treatment of sensitive, sensitized, reactive, fragile and/or weakened skin and/or mucosa.

Skin irritation reactions and allergies (delayed contact hypersensitivity or contact allergy or else contact eczema) have become a health problem in industrialized countries. The causes are as varied as the number of contact irritants and allergens that are found, for example, in metal salts, cosmetic and hygiene products, fragrances, medicines, preservatives, disinfectants, clothing, plants, etc. In this context, the number of people who say they have sensitive or reactive skin has greatly increased in the past few years. This number has gone from 30% of the population in the 1980s to around 60% today.

One of the most important causes of skin sensitivity relates to weakening of the barrier function induced, among other things, by a hereditary/acquired deficiency of intercellular lipids of the stratum corneum. Increased neurosensory activity, which is characterized by changes in epidermal nerve endings, an accumulation of neurotransmitters or a disruption in the transmission of information in the central nervous system, is also a factor that can cause increased skin sensitivity. A third, additional, cause of skin sensitivity is increased immune sensitivity, notably including a measurable increase in epidermal Langerhans cell (LC) density, and may, in the most extreme cases, lead to pathologies such as contact urticaria, irritative or allergic contact dermatitis or atopic dermatitis.

Sensitive skin polymorphism is reflected by subjective feelings such as redness, feeling of heat or warmth, tension, tingling, stinging or tightness. These unesthetic and/or uncomfortable manifestations are characteristic of sensitive skin. In the most extreme cases, irritations and even allergic reactions are also described.

Both in cosmetics and in pharmacy, it is known to reduce the sensitivity of all types of skin, especially by preventing and/or treating the inflammatory or irritating reaction. In subjects with so-called “sensitive” skin, even a minor exposure to aggressive agents or irritating conditions can be reflected by said unesthetic and/or uncomfortable cutaneous and/or mucosal manifestations which can even lead to a major inflammation or irritation reaction, which should be avoided. Current treatments for sensitive skin have the objective of making the skin more tolerant, i.e., increasing the reactivity threshold of sensitive skin, which are characterized by a lower irritability threshold to irritant reactions, for example by inhibiting the release of proinflammatory cytokines IL-6 and IL-8. In view of the number of people with skin sensitivity problems and despite the treatments currently available, it is vital to find new treatments with an effect on the factors for skin inflammation to be able to treat such skin.

The inventors have discovered that the protein extract of ungerminated, deoiled Moringa oleifera seed also had an impact on cutaneous and/or mucosal microbial flora, in particular by activating the growth of beneficial commensal strains, particularly those that fight inflammation such as S. epidermidis and/or A. lwoffi (Example 3b), as well as inhibiting the growth of opportunistic pathogenic strains such as Propionibacterium acnes (Example 3a).

The skin actually represents a complex ecosystem on which several types of microorganisms, such as bacteria and fungi, proliferate. These microorganisms constitute the cutaneous flora, also known as the cutaneous microbial flora. There are:

beneficial commensal resident flora consisting of microorganisms conventionally proliferating on healthy skin, in an ongoing manner by drawing their nutrients from the skin, and providing known benefits to the skin,

opportunist pathogenic resident flora such as Propionibacterium acnes, which normally lives on the skin but which, under certain conditions, can become virulent and therefore potentially pathogenic and

transient flora, present on the skin under abnormal conditions, for example by contact with contaminated items, which can become pathogenic in the event of proliferation.

Cutaneous commensal flora microorganisms particularly include the strain Staphylococcus epidermidis and the strain Acinetobacter lwoffii. These strains are notably found on the face in healthy skin, where they participate in maintaining the balance of cutaneous commensal flora.

In return, the strain Staphylococcus aureus can be considered as part of the potentially pathogenic cutaneous transient flora for human skin and the strain Propionibacterium acnes as being part of the opportunistic pathogenic resident flora. Staphylococcus aureus and Propionibacterium acnes may actually cause, in the event of proliferation, a microbial imbalance in the cutaneous flora, which makes it more vulnerable to infections and fungal infections, induce inflammation expressed in particular by redness, swelling or even pimples and a feeling of burning and/or localized heat accompanied by pain, pigmentation spots or scars, for example, due to acne, and therefore inhomogeneity of the complexion, and finally can cause actual infectious skin pathologies such as skin infections like boils, folliculitis, ulcers, abscesses, sycosis, impetigo, ecthyma, erysipelas or acne. Moreover, a disruption in commensal flora may also cause fungal infections of the skin, such as candidiasis. Disruption of the oral mucosal flora, in particular of the gingival mucosa, may cause gingivitis. Cosmetic or dermatological solutions are already known for acting on the cutaneous microbial flora. However, their actions are often antiseptic. These modes of action are therefore not targeted because they are not directed against a specific microorganism strain. There are solutions which make it possible to act on the cutaneous microbial flora by targeting a group such as bacteria, in the field of pharmaceutical ingredients. Examples include antibiotics. However, antibiotics have the disadvantage of not always being tolerated, especially by oral administration, and also cause the development of resistance. Moreover, antibiotics generally act not only on pathogenic microbial flora, but also on commensal microbial flora, which can lead to fungal infections. Consequently, there is a great need in the field of cosmetics and dermatology to provide ingredients which act on the cutaneous microbial flora, especially by targeted action on a particular microbial strain or by protection of commensal flora, which ingredients are readily available and do not have the disadvantages or side effects previously described.

The present invention therefore concerns the cosmetic use of a protein extract of ungerminated, deoiled Moringa oleifera seed, advantageously of a protein extract of ungerminated, delipidated seed, in particular a protein extract of deoiled oilcake, more particularly delipidated oilcake, from ungerminated seeds, more advantageously kernels of ungerminated seeds of Moringa oleifera, for the treatment of sensitive, sensitized, fragile and/or weakened skin and/or mucosa.

Generally, sensitive skin and/or mucosa can be defined as skin which, by nature, does not tolerate aggressive agents well, especially environmental agents such as pollutants, climate factors (wind, cold, heat), UV exposure, emotional factors, especially stress and/or chemical agents (heavy metals, detergents, compounds contained in cosmetic treatments such as fragrances, preservatives, alcohols, pH, AHA or dermatological treatments, such as vitamin A acid) and/or aggressive conditions, including perspiration and mechanical aggression such as waxing, shaving, rubbing and even water, especially hard water. Sensitive skin is not pathological skin, unlike allergic skin. Nevertheless, it may react to aggressive agents and/or conditions by unesthetic and/or uncomfortable cutaneous and/or mucosal manifestations such as stinging, feeling of heat or warmth, tension, tingling, tightness and redness. Thus the “sensitive skin” character may be estimated by the subject themselves with subjective cutaneous sensations or by the dermatologist with objective cutaneous reactions.

Unesthetic and uncomfortable manifestations may be generalized to the entire body, but most of the time they can have well-defined locations such as, for example, the scalp, face, skin folds, buttocks in infants, etc. It can therefore be question of areas of sensitive skin and/or mucosa.

Likewise, sensitized skin is skin momentarily made sensitive, therefore nonpathological as such.

Reactive or hyperreactive or intolerant or irritable skin is skin whose tolerance threshold has decreased and which reacts excessively.

A fragile or weakened skin, i.e. skin made momentarily fragile, is skin whose barrier function is weakened. This may be linked to the status of the individual; elderly people and infants have more fragile skin, for example. This state may result from chemical or physical aggression (abrasion, rubbing, cuts).

The uncomfortable and unesthetic manifestations of sensitive, fragile and/or weakened skin are the same as for sensitive skin, without these manifestations and/or skin conditions being considered to involve the prevention and/or treatment of a pathology. For the purposes of the present invention, “cosmetic” is intended to mean a non-pharmaceutical, non-therapeutic use, which is not intended for prevention and/or treatment of skin and/or mucosa qualified as pathological by a specialist in the field, such as a dermatologist. It is therefore a use on healthy skin and/or mucosa.

“Healthy skin and/or mucosa” is intended to mean all or part of a healthy area of skin including the scalp and/or mucosa, notably human, therefore with no infections, scars, skin diseases or conditions such as candidiasis, impetigo, psoriasis, eczema, acne, ichthyosis, gingivitis or dermatitis or wounds or injuries or canker sores or ulceration or burning and/or other dermatoses, or aphthoses or inflammation or irritation.

For the purposes of the present invention, “skin” is intended to mean the skin of all or part of the body, particularly human, chosen from the legs, feet, underarms, hands, thighs, stomach, chest, neck, arms, torso, back, labial mucosa, face and/or scalp, advantageously the chest and/or face, even more advantageously the face.

For the purposes of the present invention, “mucosa” is intended to mean the ocular mucosa, vaginal mucosa, urogenital mucosa and/or oral mucosa, notably oral, labial mucosa and/or gingival mucosa, preferentially ocular and/or oral mucosa and more preferentially gingival, labial and/or ocular mucosa, still more preferentially gingival mucosa.

For the purposes of the present invention, “treatment of sensitive, sensitized, fragile and/or weakened skin and/or mucosa” is intended to mean the fact of reducing the reactivity thereof so as to make them less sensitive and/or fragile, for example by reducing and/or inhibiting the release of cytokines IL6 and/or IL8 relative to sensitive, sensitized, fragile and/or weakened skin and/or mucosa untreated by the extract according to the invention, in particular such as described in Example 2.

The protein extract of ungerminated, deoiled Moringa oleifera seed according to the invention is topically and/or orally acceptable. For the purposes of the present invention, “topically acceptable” is intended to mean an ingredient suitable for topical application that is non-toxic and non-irritant for the skin and/or mucosa, that does not induce an allergic response and that is not chemically unstable.

For the purposes of the present invention, “orally acceptable” is intended to mean an ingredient suitable for oral administration that is non-toxic, that does not induce an allergic response and that is not chemically unstable.

Thus, advantageously, the use of the extract according to the invention is for preventing and/or treating the unesthetic and/or unpleasant and/or uncomfortable manifestations of sensitive, sensitized, fragile and/or weakened skin and/or mucosa, advantageously chosen from redness, the feeling of heat or warmth, tension, tingling, stinging, tightness and a mixture of these manifestations. It provides a feeling of comfort to the skin and/or mucosa.

The present invention also concerns the cosmetic use of a protein extract of ungerminated, deoiled Moringa oleifera seed, advantageously a protein extract of ungerminated, delipidated seed, in particular a protein extract of deoiled oilcake, more particularly delipidated oilcake, of ungerminated seeds, more advantageously ungerminated seed kernels, of Moringa oleifera, for increasing and/or protecting and/or maintaining the beneficial commensal flora on the skin and/or mucosa, in particular beneficial commensal bacterial flora, particularly chosen from the group made up of Staphylococcus epidermidis, Acinetobacter lwoffi and mixtures thereof.

Therefore, for the purposes of the present invention, “commensal flora or strain” is intended to mean a strain or flora that is beneficial to the skin and/or the mucosa and which is not, or does not become, pathogenic for the skin and/or the mucosa.

For the purposes of the present invention, “maintaining and/or protecting beneficial commensal flora on the skin and/or mucosa” is intended to mean maintaining constant the content, in the skin and/or the mucosa, of one or more commensal microorganism strains, chosen from the group made up of fungi, yeasts and bacteria, preferentially bacteria, present on or supplied to the skin or the mucosa, especially human, the action of which is beneficial to the skin and/or the mucosa, such as Staphylococcus hominis, S. warneri, S. capitis, S. epidermidis, Acinetobacter lwoffii, preferentially Staphylococcus epidermidis and/or Acinetobacter lwoffi.

For the purposes of the present invention, “increasing the beneficial commensal flora on the skin and/or mucosa” is intended to mean increasing the growth of beneficial commensal flora on the skin and/or mucosa. Several methods may be used to measure the content of microorganism strains in the skin and/or mucosa, including counting the colonies present on the skin or mucosa, in-vitro measurement by optical density after recovering samples containing the strains or measurement by PCR. Advantageously, the microorganism content is measured in vitro by optical density after recovery of samples containing the strains as exemplified in Example 3b.

According to the invention, the use of the protein extract of ungerminated, deoiled Moringa oleifera seed according to the invention is not to improve the barrier function of the skin nor to increase hydration of the skin and/or mucosa, nor for treatment of dry skin, nor to soothe the skin and/or the mucosa, in particular the dry skin and/or mucosa, nor to soften and/or reduce the feeling of fatigue of the skin and/or of the mucosa.

In particular, the use of the protein extract of ungerminated, deoiled Moringa oleifera seed according to the invention is not to protect the skin and/or the mucosa from pollution, more particularly urban pollution (such as heavy metals, exhaust gases and/or cigarette smoke) and advantageously from damage caused by pollution, and/or to purify the skin and/or the mucosa by eliminating the particles that make the skin and/or the mucosa impure, such as pollution particles (for example coal particles).

Thus, advantageously, the cosmetic (and therefore not therapeutic) use according to the invention is for preventing and/or reducing and/or eliminating the unesthetic and/or uncomfortable effects of the skin and/or mucosa whose beneficial commensal flora is altered, in particular for preventing and/or reducing and/or delaying the secretion of sebum and its unesthetic and/or unpleasant and/or uncomfortable manifestations, in particular for preventing and/or reducing and/or delaying the formation of blackheads and/or comedogenesis and/or the shiny appearance of the skin, and/or for maintaining and/or improving the homogeneity of the complexion of the skin and/or the mucosa, for example by elimination and/or reduction of redness and/or irregularities, and/or for preventing and/or treating the feeling of warmth and/or heat on the skin and/or the mucosa and/or for preventing and/or reducing head hair loss and/or body hair loss, and/or for preventing and/or reducing dandruff.

For the purposes of the present invention “maintaining and/or improving the homogeneity of the complexion of the skin and/or the mucosa” is intended to mean preventing and/or reducing and/or treating the complexion imperfections, such as skin redness or irregularities, so as to make the complexion of the skin and/or the mucosa more homogenous and therefore less dull and more luminous, and/or reduce the red appearance of the skin by giving it a healthy and/or nourished appearance, and therefore a healthy glow. Complexion homogeneity may be measured, for example, by chromometry or image analysis. This latter in-vivo measurement method consists of taking high-resolution photographs in crossed polarized configuration of volunteers' faces taken at 45° before and after application of the product tested. On the basis of these digital photographs, an image analysis allows extracting and quantifying specific parameters (for example: L*, a*, b*, C,)h° related to the color, brightness, the homogeneity, and the texture of the skin.

For the purposes of the present invention “skin and/or mucosa whose beneficial commensal flora is altered” is intended to mean skin and/or mucosa whose beneficial commensal flora content, in particular the content of Staphylococcus epidermidis and/or Acinetobacter lwoffii, is less than the beneficial commensal flora content, in particular the content of Staphylococcus epidermidis and/or Acinetobacter lwoffi measured initially, before the alteration or on another, unaltered part of the body.

In one advantageous embodiment, the beneficial commensal flora is a microorganism chosen from the group made up of Staphylococcus epidermidis, Acinetobacter lwoffii and mixtures thereof.

In another advantageous embodiment, the protein extract of ungerminated, deoiled Moringa oleifera seed is applied topically, advantageously to specific parts and/or areas of the body chosen from the legs, feet, underarms, hands, neck, chest, stomach, arms, thighs, hips, buttocks, waist, crotch, groin, torso, back, labial mucosa, face and/or scalp and/or oral and/or gingival mucosa, especially shaved areas, or areas of maceration such as an infant's bottom, the skinfolds such as the underarms, the back of the elbows, the back of the knees, the buttocks, the crotch, the groin, the neck, and/or the corner of the lips and/or the excessively cleaned areas.

For the purposes of the present invention, “topically” is intended to mean the application of the protein extract of ungerminated, deoiled Moringa oleifera seed and/or the composition and/or the ingredient according to the invention on the surface of the skin and/or the mucosa, notably by direct application or spraying.

In another advantageous embodiment, the protein extract of ungerminated, deoiled Moringa oleifera seed is administered orally, in particular for purposes of treating the gingival mucosa. Thus, advantageously, the protein extract of ungerminated, deoiled Moringa oleifera seed or the cosmetic ingredient comprising same is found in the form of a cosmetic composition intended for oral administration, in particular on the gingival mucosa, also comprising an appropriate cosmetic vehicle.

For the purposes of the present invention, “cosmetic and/or pharmaceutical ingredient(s)” is intended to mean one or more plant extracts and/or one or more natural or synthetic molecules and/or mixtures thereof intended for cosmetic and/or pharmaceutical application. Cosmetic ingredients are defined by the International Nomenclature of Cosmetic Ingredients (INCI).

For the purposes of the present invention, the term “appropriate cosmetic or pharmaceutical vehicle” means that the composition or the components thereof are suitable for use in contact with the human skin and/or mucosa without any undue toxicity, incompatibility, instability, allergic response, or equivalents thereof.

The extract according to the invention may be obtained by various extraction methods known to those skilled in the art, advantageously chosen from maceration, with or without stirring, hot decoction, grinding including ultrasonic grinding or using a blender. Preferentially, the extraction is done by maceration, more advantageously with stirring.

The extraction may be conducted at a temperature of 4° C. to 300° C., preferentially 20° C. to 80° C., given that 20° C. is room temperature, advantageously between 20 and 25° C. The extraction will be conducted for a period of 30 minutes to 12 hours, preferentially for a period of 1 hour to 5 hours, more preferentially for a period of 1 hour to 2 hours. Very advantageously, the extraction will be carried out for a period of 1 hour.

The extract according to the invention may be obtained by extraction in a protic polar solvent, advantageously chosen from the group made up of water, an alcohol, a glycol, a polyol, a water/alcohol mixture, from 99/1 to 1/99 (w/w), a water/glycol mixture from 99/1 to 1/99 (w/w) and a water/polyol mixture from 99/1 to 1/99 (w/w) (such as water mixed with ethanol, glycerol and/or butylene glycol and/or other glycols such as xylitol and/or propanediol, etc.), advantageously in water as sole solvent.

In particular, the extract is obtained by aqueous extraction.

For the purpose of the present invention, “extract obtained by aqueous extraction” is intended to mean any extract obtained by extraction with an aqueous solution containing more than 60% by weight, advantageously at least 70% by weight, in particular at least 80% by weight, more particularly at least 90% by weight, particularly at least 95% by weight, of water relative to the total weight of the aqueous solution, even more advantageously not containing glycol and in particular not containing alcohol, more particularly only containing water.

The extract may be obtained from an amount of 0.1% to 20%, fresh or dry matter, preferentially dry, advantageously 1% to 10%, more advantageously 5% to 10%, very advantageously from an amount of 10% by weight of dry matter, of ungerminated, delipidated seeds of the Moringa oleifera plant, relative to the total weight of ungerminated, delipidated plant seeds and solvent.

In one advantageous embodiment, the protein extract of ungerminated, deoiled Moringa oleifera seed is such as described in patent EP1064008 and is sold by BASF under the trade name Purisoft®.

In particular, it is a water-soluble extract, more particularly obtained by extraction in a protic polar solvent, advantageously in water.

The extraction method is advantageously the one described in patent EP1064008 and, in particular, comprises the following steps:

a)—extraction of oil from the Moringa oleifera seeds, advantageously using a press or by reflux extraction with a nonpolar solvent such as hexane;

b)—extraction of the deoiled oilcake or flour obtained in step a) with a protic polar solvent, in particular chosen from alcohols, polyols, glycols, water and mixture thereof in any proportion, advantageously with an aqueous, i.e., water-based solvent, even more advantageously with water as sole solvent, so as to obtain a crude extract, advantageously as described in Example 1a.

Advantageously, the extraction is conducted at room temperature.

The aqueous solvent may be a saline solution at various pH or a buffered medium, more advantageously at a pH comprised between 4 and 8.

The method may also include an additional step c) after step b) of precipitating the crude extract by modification of the pH of the crude aqueous extract obtained in step b) so as to obtain a basic pH, in particular greater than 8, more advantageously greater than 11, for example using NaOH, and recovery of the precipitate obtained, advantageously such as described in Example 1b.

The precipitate may then be washed and solubilized in water so as to eliminate insoluble components and obtain an extract in the form of a protein concentrate.

The method may also comprise, after precipitation step c), an additional decantation step d) so as to allow better precipitation of proteins, for example such as described in Example 1c. In particular, this step is implemented at a temperature below room temperature, advantageously 4° C., more advantageously for at least 6 hours, in particular overnight.

Instead of steps c) and d), a step e) can be implemented after step b) comprising contacting the crude aqueous extract obtained in step b) with carboxymethylcellulose, advantageously for 1 hour, in particular at room temperature, so as to load it with the protein extract. This step may be followed by a step f) of contacting the loaded carboxymethylcellulose obtained in step e) with an aqueous saline solution, advantageously an NaCl solution, having a pH greater than 7, advantageously 7.5, and then recovering the eluate thus obtained, which is a partially purified extract.

The protein extract of ungerminated, deoiled Moringa oleifera seed is advantageously an extract of hulled seeds, that is to say advantageously an extract of the seed kernel only (without its casing or shell).

The method may thus comprise a step before step a) of hulling the seeds.

Finally, the extracts obtained (crude extract, protein concentrate and/or partially or totally purified extract) can be centrifuged and/or filtered and/or distilled, so as to recover the water-soluble fraction and be in liquid form. Preferentially, the supernatant obtained after centrifugation is filtered, advantageously at a cut-off threshold of 0.45 μm. Additional decolorizing and/or deodorizing steps can be carried out on the extracts at any stage of the extraction and according to the techniques known to those skilled in the art. In particular, the extract may be decolorized with activated charcoal.

According to one particular embodiment, the extract according to the invention is obtained by extraction of oilcakes of ungerminated, in particular deoiled, more advantageously delipidated seeds, in a saline aqueous solution at a pH of approximately 5 followed by elimination of high molecular weight proteins. The supernatant is recovered and constitutes an extract according to the invention.

The extracts may also be concentrated by evaporation of the solvent or dried, for example by freeze-drying or by spray-drying. The extracts will then be in powder form. In one particular embodiment of the invention, in particular for use thereof in dermatology, the Moringa oleifera extract obtained will be sterilized.

In one particularly advantageous embodiment, the protein extract of ungerminated, deoiled Moringa oleifera seed contains, on the basis of the dry extract, a protein content, particularly of native proteins, comprised between 0.01 and 100% by weight, advantageously at least 25% by weight, in particular at least 40% by weight, more particularly at least 45% by weight.

The proteins of the protein extract according to the invention advantageously have a molecular weight comprised between 6500 and 13000 Da, advantageously between 7100 and 11000 Da, measured by chromatography.

In particular, the protein extract of ungerminated, deoiled Moringa oleifera seed does not contain alkaloids (such as, for example, spirochin), Pterygospermin, isothiocyanates (such as, for example, 4-(2-L-rhamnosyloxy)benzyl isothiocyanate) or kaempferol.

The extract according to the invention may be used in the form of a cosmetic or pharmaceutical ingredient intended to be incorporated into a cosmetic or pharmaceutical composition, and also comprising an appropriate cosmetic or pharmaceutical vehicle.

In this case, the extract according to the invention is in another embodiment preferentially solubilized in and/or diluted in a solvent, particularly polar, such as water, advantageously also comprising glycerine as in the product sold under the name Purisoft® and, in particular, such as described in Example 4a).

The extract according to the invention in another embodiment may be spray dried onto a spray drying support such as, for example, maltodextrin and be in the form of a powder, in particular such as described in Example 4b).

Advantageously, when the ingredient is in the liquid form such as the one described in Example 4a), the extract according to the invention is present in the ingredient at a content comprised between 0.01 and 10% by weight of dry matter relative to the total weight of the ingredient, advantageously between 1 and 5% by weight.

Advantageously, when the ingredient is in the solid form, particularly powder, such as the one described in Example 4b), the extract according to the invention is present in the ingredient at a content comprised between 10 and 60% by weight of dry matter relative to the total weight of the ingredient, advantageously between 30 and 50% by weight.

The extract according to the invention optionally in the form of a cosmetic or dermatological ingredient may also be found in the form of a cosmetic or pharmaceutical composition, advantageously intended for topical or oral administration, in particular topical, preferentially cutaneous, also comprising an appropriate cosmetic or pharmaceutical vehicle.

The cosmetic or pharmaceutical, in particular dermatological, ingredient in the liquid form and particularly the one of Example 4a), may be used in a cosmetic or pharmaceutical, in particular dermatological, composition, preferentially at a content by weight of dry matter relative to the total weight of the composition comprised between 0.01 and 10%, advantageously between 0.1 and 5%, in particular between 1 and 3%.

The cosmetic or pharmaceutical, in particular dermatological, ingredient in the solid form and particularly the one of Example 4b), may be used in a cosmetic or pharmaceutical, in particular dermatological, composition, preferentially at a content by weight of dry matter relative to the total weight of the composition comprised between 0.001 and 5%, advantageously between 0.01 and 1%.

In one embodiment of the invention, the extract will be comprised in the cosmetic or pharmaceutical composition in a content comprised between 0.0001% and 20% by weight of dry matter relative to the total weight of the composition, preferentially between 0.001% and 10% by weight, advantageously between 0.01 and 5% by weight. The compositions according to the invention may contain any appropriate solvent and/or any appropriate vehicle and/or any appropriate excipient, optionally in combination with other compounds of interest. They may, in particular, contain a cosmetically or dermatologically acceptable excipient chosen from surfactants, preservatives, buffering agents, blowing agents, chelating agents, biocides, denaturants, opacifying agents, pH adjusters, reducing agents, stabilizing agents, emulsifiers, thickeners, gelling agents, film-forming polymers, solvents, fillers, bactericides, odor absorbers, mattifying agents, conditioners, texturizing agents, gloss agents, pigments, dyes, fragrances and chemical or mineral sunscreens, trace elements, essential oils, sweeteners, or taste modifiers. These combinations are also covered by the present invention. The CTFA Cosmetic Ingredient Handbook, Second Edition (1992) describes different cosmetic and pharmaceutical ingredients commonly used in the cosmetics and pharmaceutical industry, which are suitable, in particular, for oral administration and/or topical use.

Advantageously, the excipient(s) are chosen from the group comprising polyglycerols, esters, cellulose polymers and derivatives, lanolin derivatives, phospholipids, lactoferrins, lactoperoxidases, sucrose-based stabilizers, vitamin E and its derivatives, xanthan gums, natural and synthetic waxes, vegetable oils, triglycerides, unsaponifiables, phytosterols, silicones, protein hydrolysates, betaines, aminoxides, plant extracts, saccharose esters, titanium dioxides, glycines, and parabens, and more preferably from the group consisting of steareth-2, steareth-21, glycol-15 stearyl ether, cetearyl alcohol, phenoxyethanol, methylparaben, ethylparaben, propylparaben, butylparaben, butylene glycol, caprylyl glycol, natural tocopherols, glycerin, dihydroxycetyl sodium phosphate, isopropyl hydroxyketyl ether, glycol stearate, triisononanoine, octyl cocoate, polyacrylamide, isoparaffin, laureth-7, a carbomer, propylene glycol, hexylene glycol, glycerol, bisabolol, a dimethicone, sodium hydroxide, PEG 30-dipolyhydroxysterate, capric/caprylic triglycerides, cetearyl octanoate, dibutyl adipate, grape seed oil, jojoba oil, magnesium sulfate, EDTA, a cyclomethicone, xanthan gum, citric acid, sodium lauryl sulfate, waxes and mineral oils, isostearyl isostearate, propylene glycol dipelargonate, propylene glycol isostearate, PEG 8, beeswax, glycerides of hydrogenated palm heart oil, lanolin oil, sesame oil, cetyl lactate, lanolin alcohol, castor oil, titanium dioxide, lactose, saccharose, low density polyethylene, an isotonic saline solution, and mixtures thereof. The cosmetic or pharmaceutical composition or the extract according to the invention, optionally in the form of a cosmetic or pharmaceutical ingredient, may be in any of the galenical forms conventionally used for topical application or oral administration, in particular topical administration such as liquid or solid forms or in the form of pressurized liquid. They may particularly be formulated in the form of an aqueous or oily solution, a cream or an aqueous gel or an oily gel, especially in a pot or tube, especially a shower gel, a shampoo, a milk, an emulsion, a hydrogel, a microemulsion or a nanoemulsion, especially oil-in-water or water-in-oil or multiple or silicone-based emulsion, a serum, a lotion, especially in a glass or plastic bottle or measuring bottle or aerosol bottle, a vial, a liquid soap, a paste, a dermatological bar, an ointment, a foam, an aerosol, a mask, a patch, an anhydrous product, which is preferably liquid, pasty or solid, for example in the form of a rod in particular in stick form, or powders, in particular face powder. In particular, the composition is in the form of a serum, a lotion, a cream, a milk, an ointment, a paste, a foam, an emulsion, a hydrogel, a shower gel, a mask, a stick, a patch, or face powders, advantageously a cream or a lotion.

In the case of oral administration, the cosmetic or pharmaceutical composition or the extract according to the invention optionally in the form of cosmetic or pharmaceutical ingredient, may be present in the form of a dentifrice, a toothpaste, a mouthwash, a lotion, an aerosol, a gel, a lozenge, an orodispersible tablet, chewing gum or a mucoadhesive composition.

The cosmetic or pharmaceutical composition may also comprise other active ingredients in the treatment of the skin and/or of the mucosa of the sensitive, sensitized, reactive, fragile and/or weakened skin and/or mucosa and/or having an effect on the increase and/or protection and/or maintenance of the beneficial commensal flora in the skin and/or the mucosa, inducing a complementary or synergistic effect with the extract according to the invention, chosen, for example, from

a combination of sodium hyaluronate, pullulan and sodium alginate, especially marketed in a formulation containing serine, trehalose, urea and glycerin under the name PatcH2O™ by the applicant;

the cosmetic agents intended for sensitive skin care such as a plant extract of Cestrum latifolium such as described in application WO2009/112590, for example marketed under the name Symbiocell™ by the applicant, a butter extracted from the fruit of the Irvingia gabonensis tree marketed under the name Irwinol™ by the applicant, a root extract of Eperua falcata marketed under the name Eperuline™, an N-acetyl-L-Tyrosyl-L-Prolyl-L-Phenylalanyl-L-Phenylalaninamide peptide (INCI: acetyl tetrapeptide 15) sold under the name Skinasensyl™ by the applicant.

The cosmetic composition may also contain one or more other ingredients active on the cutaneous and/or mucosal microbial flora and/or active on the barrier function of the skin, in particular moisturizing and/or soothing active agents, including an oligosaccharide obtained by enzymatic synthesis marketed by the company Solabia under the name BioEcolia™ or an alpha-glucooligosaccharide complex marketed by the same company under the name Ecoskin™, an extract of Alisma plantago-aquatica, an extract of Argania spinosa (Lipofructyl™ Argan), a ceramide mixture (Sphingoceryl™ VEG), purifying Boldo extracts (Betapur™), products based on inulin or fructooligosaccharides, extracts of bifidobacteria or else an extract of Orthosiphon stamineus to combat oily skin (MAT-XS™ Bright), a natural honey extract marketed by the applicant under the name of Melhydran™ for its moisturizing property, a flax extract marketed under the name Oligolin™ by the applicant, a yeast extract modified by biotechnology and marketed by the applicant under the name Relipidium™, a Pueraria lobata root extract marketed under the name Inhipase™ by the applicant, a beta-glucan derivative derived from baker's yeast marketed by Mibelle under the name CM-Glucan Forte™ and/or an extract of Mirabilis jalapa marketed under the name Pacifeel™ by Sederma.

Advantageously, the object of the invention is also a cosmetic treatment method for the treatment of sensitive, sensitized, fragile and/or weakened skin and/or mucosa for preventing and/or treating its unesthetic and/or unpleasant and/or uncomfortable manifestations, in particular chosen from redness, the feeling of heat or warmth or tension, tingling, stinging, tightness and a mixture of these manifestations, and/or for increasing and/or protecting and/or maintaining the beneficial commensal flora in the skin and/or the mucosa, in particular for preventing and/or reducing and/or delaying the unesthetic and/or unpleasant and/or uncomfortable manifestations of the skin and/or mucosa whose beneficial commensal flora is altered, particularly for preventing and/or reducing and/or delaying the secretion of sebum and its unesthetic and/or unpleasant and/or uncomfortable manifestations such as for preventing and/or reducing and/or delaying the formation of blackheads and/or comedogenesis and/or the shiny appearance of the skin, and/or for maintaining and/or improving the homogeneity of the complexion of the skin and/or the mucosa and/or reducing redness, and/or for preventing and/or treating the feeling of warmth and/or heat of the skin and/or the mucosa, and/or for preventing and/or reducing head hair loss and/or body hair loss and/or for preventing and/or reducing dandruff, said skin and/or mucosa being healthy skin and/or mucosa of an individual who needs/who wants this treatment, comprising the following steps:
a) Identification in the individual of an area of sensitive or sensitized, fragile or weakened skin and/or mucosa that one wishes to treat and/or one wishes to prevent and/or treat its unesthetic and/or unpleasant and/or uncomfortable manifestations, in particular chosen from redness, the feeling of heat or warmth or tension, tingling, stinging, tightness and a mixture of these manifestations, and/or for which one wishes to increase and/or protect and/or maintain the beneficial commensal flora in the skin and/or the mucosa, in particular for preventing and/or reducing and/or delaying the unesthetic and/or unpleasant and/or uncomfortable manifestations of the skin and/or mucosa whose beneficial commensal flora is altered, particularly for preventing and/or reducing and/or delaying the secretion of sebum and its unesthetic and/or unpleasant and/or uncomfortable manifestations such as for preventing and/or reducing and/or retarding the formation of blackheads and/or comedogenesis and/or the shiny appearance of the skin, and/or for maintaining and/or improving the homogeneity of the complexion of the skin and/or the mucosa and/or reducing redness, and/or for preventing and/or treating the feeling of warmth and/or heat of the skin and/or the mucosa, and/or preventing and/or reducing head hair loss and/or body hair loss and/or preventing and/or reducing dandruff, said skin and/or mucosa being healthy skin and/or mucosa, and
b) The topical application on this area of skin or the oral administration of a cosmetic composition containing the protein extract of ungerminated, deoiled Moringa oleifera seed according to the invention in an effective amount for the treatment of sensitized, fragile and/or weakened skin and/or mucosa for preventing and/or treating its unesthetic manifestations, in particular chosen from redness, the feeling of heat or warmth or tension, tingling, stinging, tightness and a mixture of these manifestations, and/or for increasing and/or protecting and/or maintaining the beneficial commensal flora in the skin and/or the mucosa, in particular for preventing and/or reducing and/or delaying the unesthetic and/or unpleasant and/or uncomfortable manifestations of the skin and/or mucosa whose beneficial commensal flora is altered, particularly for preventing and/or reducing and/or delaying the secretion of sebum and its unesthetic and/or unpleasant and/or uncomfortable manifestations such as for preventing and/or reducing and/or delaying the formation of blackheads and/or comedogenesis and/or the shiny appearance of the skin, and/or maintaining and/or improving the homogeneity of the complexion of the skin and/or the mucosa and/or reducing redness, and/or for preventing and/or treating the feeling of warmth and/or heat of the skin and/or the mucosa, and/or preventing and/or reducing head hair loss and/or body hair loss and/or for preventing and/or reducing dandruff, said skin and/or mucosa being healthy skin and/or mucosa.

The present invention also relates to a protein extract of ungerminated, deoiled Moringa oleifera seed, advantageously a protein extract of ungerminated, delipidated seed, in particular a protein extract of deoiled oilcake, more particularly delipidated oilcake, of ungerminated seeds, more advantageously of ungerminated seed kernels, of Moringa oleifera, for its use in the treatment of reactive, hyperreactive, intolerant and/or irritated skin and/or mucosa and/or in the treatment and/or prevention and/or reduction of the occurrence of pathologies related to skin and/or mucosa that are sensitive, sensitized, reactive, fragile, weakened, intolerant, hyperreactive and/or irritated, such as contact urticaria, irritative or allergic contact dermatitis, eczema, psoriasis, seborrheic or atopic dermatitis, and/or in the treatment and/or prevention of inflammation and/or irritation, particularly caused by Staphylococcus aureus, and/or in the treatment and/or prevention of erythema, particularly infant diaper rash and/or in the treatment of gingivitis.

The present invention also relates to a protein extract of ungerminated, deoiled Moringa oleifera seed, advantageously an ungerminated, delipidated seed protein extract, in particular a protein extract of deoiled oilcake, more particularly delipidated oilcake, of ungerminated seeds, more advantageously of ungerminated Moringa oleifera seed kernels, for its use in the treatment and/or prevention and/or reduction of the occurrence of pathologies related to an alteration of the cutaneous and/or mucosal beneficial commensal flora, advantageously involving a decrease of the content of beneficial commensal cutaneous and/or mucosal microorganisms and/or an increase in the content of pathogenic microorganisms, preferably in pathogenic bacteria, in particular Staphylococcus aureus and/or Propionibacterium acnes, advantageously pathologies chosen from the group consisting of infections, especially bacterial, of the skin and/or mucosa, ulcers, herpes, boils, folliculitis, abscesses, sycosis, impetigo, ecthyma erysipelas, acne, fungal infections such as candidiasis or dermatophytosis, such as ringworm and/or scabies and/or in the treatment and/or prevention of wound infections, and/or in the prevention of pigment spots and/or acne scars.

Moreover, for the purposes of the present invention, the term “pathogenic microorganism” is intended to mean a microorganism present on the skin and/or the mucosa in a non-permanent (transient) manner or a microorganism which normally lives on the skin but which under certain conditions may become virulent. and therefore potentially pathogenic (opportunistic resident pathogen) and inducing or capable of inducing non-pathological alterations of the skin and/or the mucosa such as skin imperfections such as, for example, redness, dandruff and/or head hair and/or body hair loss, swelling or pimples and/or a burning sensation and/or localized heat accompanied by pain, pigmentation spots or scars for example due to acne and therefore an inhomogeneity of the complexion, and may be involved in actual skin pathologies including skin infections, such as boils, folliculitis, abscesses, ulcers, sycosis, ecthyma, erysipelas, acne or impetigo, even pathological conditions, such as fungal infections (such as candidiasis or dermatophytosis) such as scabies, ringworm and fungal infections caused by Candida albicans, Malassezia, Streptococci, Propionibacterium acnes, Staphylococci and, especially, Staphylococcus aureus.

Finally, the present invention relates to a cosmetic care method characterized in that it comprises the application to at least one relevant area of the skin and/or the mucosa of the face or body, and/or oral administration of the extract according to the present invention, optionally in the form of a cosmetic ingredient or a cosmetic composition comprising same, as active agent, the extract according to the invention as defined above, for treating sensitive, sensitized, fragile and/or weakened skin and/or mucosa, and/or for increasing and/or protecting and/or maintaining the beneficial commensal flora in the skin and/or the mucosa and advantageously for treating the unesthetic and/or unpleasant and/or uncomfortable manifestations of the skin and/or mucosa that are sensitive, sensitized, fragile, and/or weakened, advantageously chosen from among redness, the feeling of heat or warmth, or tension, stinging, tingling, tightness and a mixture of these manifestations and/or for preventing and/or reducing and/or delaying the unesthetic and/or unpleasant and/or uncomfortable manifestations of the skin and/or mucosa whose commensal beneficial flora is altered, in particular for preventing and/or reducing and/or delaying the secretion of sebum and its unesthetic and/or unpleasant and/or uncomfortable manifestations such as for preventing and/or reducing and/or delaying the formation of blackheads and/or comedogenesis and/or the shiny appearance of the skin, and/or maintaining and/or improving the homogeneity of the complexion of the skin and/or the mucosa and/or reducing redness, and/or preventing and/or treating the feeling of warmth and/or heat of the skin and/or the mucosa, and/or for preventing and/or reducing head hair loss and/or body hair loss and/or for preventing and/or reducing dandruff.

Other aims, features and advantages of the invention will emerge clearly to a person skilled in the art on reading the explanatory description, which makes reference to examples that are given purely as illustrations and shall not in any way limit the scope of the invention.

The examples form an integral part of the present invention, and any feature appearing to be novel over any prior art whatsoever, from the description taken in its entirety, including the examples, forms an integral part of the invention in its function and in its general nature.

Thus, each example has a general scope.

Moreover, in the examples and unless otherwise indicated, the temperature is expressed in degrees Celsius and the pressure is the atmospheric pressure.

### EXAMPLE 1

### Preparation of the Moringa Extract according to the Invention

The Moringa extract is prepared according to the method described in patent EP1064008, in particular as described below:

### EXAMPLE 1a

### Preparation of Extract 1a

Kernels of Moringa oleifera obtained after hulling the seeds and containing 33.4% (weight/weight) oil are delipidated by two successive extractions at reflux in hexane and after filtration, the flour is dried in an oven at 40° C. and has a residual oil content of 2.5%.

In a reactor, 200 g of delipidated flour are added to 2 litres of distilled water. After 10 minutes of stirring, the pH is adjusted to 7.5 by addition of 4N NaOH the and the extraction is then conducted for one hour at room temperature while maintaining the pH at 7.5. The insoluble component is eliminated by centrifugation for 15 min. at 5000 g.

The supernate is collected and then filtered on 0.45 μm: thus 1.77 litres of yellow filtrate are obtained, containing 4.69% of dry extract and having a protein concentration measured by the biuret technique of 21.54 g/l (or a protein purity on the basis of the dry extract of 45.92%). The extract is dehydrated by spray drying and 65.72 grams of atomisate are obtained with a protein content estimated at 54.7% (N×6.25).

If the peaks eluted between the excluded volume and the total volume of the column are considered, the chromatographic profile resulting from the analysis by gel permeation on Superose 12HR column of this extract shows a major fraction that represents 52% of the area and corresponds to a molecular weight between 7,800 and 11,000 Da. The presence of shoulders in this peak confirms the existence of several compounds and the molecular weight range is close to the one found in the literature for monomers (6,500 and 7,000 Da) and dimers (13,000 Da) of flocculant Moringa proteins.

### EXAMPLE 1b

### Preparation of Extract 1b

300 g of delipidated flour are extracted according to Example 1a so as to obtain a crude aqueous extract. The filtrate pH (2.74 litres) is adjusted to 11.8 by progressive addition of 4N NaOH. The precipitation starts around pH 8.0 (distinct clouding of the solution) and after 30 minutes the solution is centrifuged for 15 min at 5000 g. The sticky precipitate is collected (43.2 g wet) and then washed twice with 500 ml of distilled water at pH 11.8.

The precipitate is then dissolved in 270 ml of distilled water (i.e., 10% of the initial volume) and the pH of the solution is continuously adjusted to 4.5 by 6N HCl so as to allow the precipitate to solubilize (dispersion is facilitated using a device known as a Turax).

After 30 min of stirring, the mixture is centrifuged for 15 min. at 5000 g to eliminate the insoluble component and the supernatant is filtered through a Büchner funnel provided with a Whatman No. 41 filter. Thus, 260 ml of yellow and clear protein concentrate are obtained, and this concentrate is dehydrated by freeze drying.

In this way, 11.5 grams of lyophilisate are obtained with a protein content by weight of 90-95%.

The gel permeation analysis of this extract on Superose 12HR column shows a major fraction that represents 70% of the area and corresponds to a molecular weight of around 8,800 Da.

### EXAMPLE 1c

### Preparation of Extract 1c

The oil from kernels obtained by hulling Moringa oleifera seeds is extracted by pressure on a press known by the name KOMET and the oilcakes obtained are milled to obtain a homogeneous flour.

A crude extract is prepared from 1.24 kg of oilcakes according to the operating procedure described in Examples 1a and 1b.

The proteins are precipitated at pH 11.8 according to Example 1b, but an additional decantation step for one night at +4° C. is introduced in order to allow better precipitation of the proteins.

The precipitate is processed under the same conditions as in Example 1b (the pH of the precipitate reconstitution solution is 6 instead of 4.5, however).

The protein concentrate thus obtained (1.05 litres at 4.59% of dry extract) is dehydrated by spray drying and 34.6 g of atomisate are collected, or a spray drying yield on the basis of dry extract of 71.5%.

The protein content on the basis of nitrogen assay (N×6.25) is greater than 90% (approximately 95%).

### EXAMPLE 1d

### Preparation of Extract 1d

A crude extract is prepared from 150 g of oilcakes according to the operating procedure described in Examples 1a, 1b and 1c.

After filtration on 0.45 μm, 1.35 litres of clear yellow filtrate are obtained.

100 grams of Carboxymethylcellulose (CM52, WHATMAN) are equilibrated for 30 minutes in 500 ml distilled water at pH 7.5.

The mixture is filtered through a Büchner funnel provided with a WHATMAN No. 42 filter and then the cellulose is collected and again equilibrated in 500 ml of water at pH 7.5.

After elimination of the aqueous medium by filtration, the cellulose is contacted, with stirring for one hour at ambient temperature, with the aqueous extract of the Moringa oleifera kernel oilcakes.

The non-adsorbed compounds (fractions whose chromatographic profile is shown by a dashed line in FIG. 3) are eliminated by filtration through a Büchner funnel and the “loaded” cellulose is then washed twice with one litre of distilled water at pH 7.5 and then filtered through a Büchner funnel.

The cellulose is then contacted with 120 ml of a 60 g/l NaCl solution at pH 7.5 for 30 minutes.

The proteins eluted in the NaCl medium are recovered by filtration through a Büchner funnel (the chromatographic profile of the proteins adsorbed on the CM 52 and eluted in 60 g/l NaCl medium is shown in the solid line in FIG. 3).

Therefore, 110 ml of a filtrate with a dry extract content of 9.63% and with a protein concentration of 64.6 g/l (or a protein purity on the basis of the dry extract of 67%) are obtained.

The gel permeation analysis on Superose 12HR column of this extract shows a major fraction that represents 70% of the area and corresponds to a molecular weight of 7,100 Da.

The solution may be desalinated by dialysis, or by ultrafiltration and dehydrated by freeze drying, spray drying or any other appropriate means. According to Example 1d, the extract is spray dried on a maltodextrin support and formulated according to Example 4b) to be tested in the following examples.

### EXAMPLE 2

### Assessment of the Effect of the Ingredient according to the Invention on Inhibition of the Release of Proinflammatory Cytokines IL-6 and IL-8 Induced by the Opportunistic Pathogenic Bacteria S. aureus

The product tested is the one from Example 1d) formulated in the form of the ingredient according to Example 4b and used at 0.03% by weight relative to the total final medium (medium+product). Testing is done on human HacaT keratinocytes in culture.

HacaT keratinocytes are inoculated at 2×10⁶ cells/cm² in the DMEM (Dulbecco's Modified Eagle Medium) medium supplemented with 10% fetal calf serum (FCS). The cells are incubated 3 to 5 days at 37° C. in 5% CO₂ and at 95% relative humidity. The DMEM medium is then replaced by 1 mL of EMEM (Eagle's Minimum Essential Medium) medium. The product to be tested is then added in the desired concentration to the EMEM medium and everything is incubated for one additional day until the cell layer is saturated.

0.1 ml Staphylococcus aureus DSMZ 20231 ATCC 12600 bacteria solution (1.5×10⁷) is added to each well for 2 hours at 37° C., 5% CO₂, under aerobic conditions, at relative humidity saturation. Next, the media with bacteria are replaced by EMEM containing the product to be tested. Everything is incubated again for 24 hours at 37° C. and 5% CO₂.

IL8 and IL6 are assayed according to the recommendations of the ELISA kit supplier.

TABLE 2.1
Assessment of the effect of the product according to the
invention on the release of cytokine IL6 induced in keratinocytes
by the presence of S. aureus

| | Mean (%) IL6 | Standard | Significance versus |
|---|---|---|---|
| | release | deviation | S. aureus |
| Untreated control | 69 | 9 | p < 0.05 |
| Inflammation control | 100 | 9 | NA |
| (S. aureus) | | | |
| Product at 0.03% + | 5 | 2 | p < 0.05 |
| S. aureus | | | |

TABLE 2.2
Assessment of the effect of the product according to the
invention on the release of cytokine IL8 induced in keratinocytes
by the presence of S. aureus

| | Mean (%) IL8 | Standard | Significance versus |
|---|---|---|---|
| | release | deviation | S. aureus |
| Untreated control | 53 | 10 | p < 0.05 |
| Inflammation control | 100 | 15 | NA |
| (S. aureus) | | | |
| Product at 0.03% + | 3 | 2 | p < 0.05 |
| S. aureus | | | |

The product of the invention induced a decrease in the release of cytokines IL-6 and IL-8 induced by S. aureus pathogenic bacteria in keratinocytes in culture. This demonstrates the effect of the product to treat sensitive, reactive, fragile, weakened or sensitized skin, as well as its antiinflammatory effect.

### EXAMPLE 3

### Selective Effects of the Product on the Growth of Commensal Microorganisms with Regard to Opportunistic Pathogenic Microorganisms

The product of the invention has a selective action on the growth of microorganisms of the skin. The product promotes the growth of commensal microorganisms and, in particular, bacteria which help the skin to combat inflammation such as Staphylococcus epidermidis and Acinetobacter lwoffii.

Moreover, it has little effect on opportunistic pathogenic bacteria such as P. acnes responsible for inflammatory phenomena on the skin.

### EXAMPLE 3a

### Inhibition of the Growth of the Opportunistic Pathogenic Strain Propionibacterium acnes

The product tested is the one from Example 1d) formulated in the form of the ingredient of Example 4b) and used at 0.0006% and 0.006% by weight relative to the weight of the total final medium (medium and product).

The ingredients (Phenonip® for the positive control or product according to the invention) are diluted in the bacteria culture medium (brucella broth). The media containing the ingredients are then incubated 48 h at 35° C.+/−2.5° C. while respecting anaerobic conditions with an inoculum calibrated at 5×10⁵ of P. acnes ATCC 11827. The growth is estimated by measuring the optical density at 600 nm after incubation.

TABLE 3.1
Assessment of the effect of the product at different dosages
on the growth of Propionibacterium acnes

| | Mean OD at 600 nm | Standard deviation |
|---|---|---|
| Untreated control | 0.437 | 0.011 |
| Positive control | 0.1925 | 0.006 |
| (Phenonip ®) | | |
| Product at 0.0006% | 0.408 | 0.004 |
| Product at 0.006% | 0.327 | 0.057 |

### EXAMPLE 3b

### Activation of the Growth of Commensal Strains to Combat S. epidermidis and/or A. lwoffi Inflammation

The product tested is the one from Example 1d formulated in the form of an ingredient according to Example 4b) and used at 0.0006% and 0.006% by weight relative to the weight of the total final medium (medium and product).

For S. epidermidis (strain ATCC 14990): After preculture in enriched medium (Tryptic soy broth, TSB), a microplate containing the appropriate culture medium is inoculated with the bacteria at OD₆₀₀=0.05 (≈10⁷ bacteria) and incubated 24 h with the product of the invention at different concentrations. The growth is estimated by measuring the optical density at 600 nm after incubation. An untreated control is done, as is an inhibition control (sodium dodecyl sulfate or SDS).

The results show that the product according to the invention promotes the growth of the commensal microorganisms S. epidermidis and A. lwoffi.

### EXAMPLE 4

### Cosmetic or Pharmaceutical Ingredients according to the Invention containing the Moringa Extract according to the Invention

4a) A liquid cosmetic or pharmaceutical ingredient with the formulation below in percentage by weight is prepared.

| Name | Amount in % by weight |
|---|---|
| glycerin | 67 |
| Moringa extract according | 1.6 |
| to Examples 1a-1d | |
| Phenoxyethanol | 2 |
| disodium phosphate | 0.8 |
| Citric acid | 0.5 |

4b) A solid ingredient in the form of powder with the following formulation is prepared:

| Name | Amount in % by weight |
|---|---|
| Maltodextrin | 50 |
| Moringa extract according | 50 |
| to Examples 1a-1d | |

### EXAMPLE 5

### Composition according to the Invention in the Form of a Body and/or Face Lotion

| | | Amount (% by |
|---|---|---|
| Phase | Name | total weight) |
| A | Water | 69.90 |
| A | Disodium EDTA | 0.05 |
| A | Xanthan gum | 0.20 |
| B | Steareth -2 | 2.00 |
| B | Steareth-21 | 2.50 |
| B | Cetearyl alcohol | 1.00 |
| B | Propylheptyl caprylate | 15.00 |
| H | Cosmetic ingredients according | 1.50 |
| | Example 4a) containing the Moringa | |
| | extract according to the invention | |
| D | Water | qs |
| D | Sodium hydroxide (30% in solution) | 0.10 |
| E | Mixture of phenoxyethanol, | 1.25 |
| | chlorphenesin, benzoic acid, butylene | |
| | glycol and sorbic acid (Germazide ™ | |
| | PBS) | |
| F | Mixture of polyacrylate-X, | 4.00 |
| | isohexadecane and polysorbate 60 | |
| | (Sepigel ™ SMS 60) | |

The lotion is prepared by the usual methods in the field well known to those skilled in the art, by mixing the 6 phases.

### EXAMPLE 6

### Composition according to the Invention in the Form of a Body and/or Face Milk

A cosmetic product in the form of a body and/or face milk can, for example, have a weight composition made up of the following aqueous and fatty phases as indicated below.

| | | |
|---|---|---|
| Fatty phase | Isostearyl and diglyceryl succinate | 3.00 |
| | Paraffin oil | 15.00 |
| | Quaternium-18 Hectorite | 0.50 |
| | Poly(PEG-22/Dodecyl Glycol) | 1.00 |
| Aqueous phase | Magnesium sulfate | 0.80 |
| | Butylene glycol | 4.00 |
| | Protein extract of Moringa oleifera | 1.00 |
| | (according to Ex. 1a) | |
| | Distilled water | 9.00 |
| | Elestab 4112 (Laboratoires | 0.35 |
| | Sérobiologiques) | |
| | Fragrance | 0.30 |
| | Distilled water | Qs to |
| | | 100.00 |

The method for preparing the face and/or body milk consists essentially of bringing the fatty phase to 80° C., bringing the water of the aqueous phase also to 80° C. and dissolving the preservative therein (Elestab 4112), then pouring the aqueous phase into the fatty phase with turbine stirring and cooling gradually with stirring, then adding thereto, at around 50° C., the aqueous Moringa protein extract stock solution, then the fragrance and, finally, continuing the stirring until complete cooling.

### EXAMPLE 7

### Composition according to the Invention in the Form of a Face Cream

A cosmetic product in the form of a body and/or face cream can, for example, have a weight composition made up of the following aqueous and fatty phases as indicated below.

| | | |
|---|---|---|
| Fatty phase | Ceteareth 25 | 2.00 |
| | Ceteareth 6 (and) Stearyl Alcohol | 1.00 |
| | Cetyl alcohol | 4.00 |
| | Glycerol stearate | 4.00 |
| | Petrolatum | 5.00 |
| | Caprylic/capric triglycerides | 5.00 |
| Aqueous phase | Glycerin | 10.00 |
| | Moringa oleifera proteins | 1.50 |
| | (prepared according to Ex. 1b) | |
| | Distilled water | 8.50 |
| | Elestab 4112 (Laboratoires | 0.40 |
| | Sérobiologiques) | |
| | Fragrance | 0.30 |
| | Distilled water | Qs to |
| | | 100.00 |

The method for preparing the face cream consists essentially of bringing the fatty phase to 80° C., bringing the aqueous phase also to 80° C. and dissolving the Elestab 4112 therein, separately preparing the Moringa oleifera protein extract stock solution of, pouring the fatty phase into the aqueous phase with turbine stirring, then, at approximately 50° C., introducing the Moringa extract stock solution and finally continuing to stir until cool.

1. A cosmetic method comprising applying a protein extract of ungerminated, deoiled Moringa oleifera seed for the treatment of sensitive, sensitized, fragile and/or weakened skin and/or mucosa for preventing and/or treating the unesthetic and/or unpleasant and/or uncomfortable manifestations of sensitive, sensitized, fragile and/or weakened skin and/or mucosa, said skin and/or mucosa being healthy skin and/or mucosa.

2. The cosmetic method as claimed in claim 1, characterized in that the unesthetic and/or unpleasant and/or uncomfortable manifestations of sensitive, sensitized, fragile and/or weakened skin and/or mucosa are chosen from redness, the feeling of heat or warmth or tension, tingling, stinging, tightness and a mixture of these manifestations

3. The cosmetic method as claimed in claim 1, characterized in that the use of the protein extract of ungerminated, deoiled Moringa oleifera seed is also for increasing and/or protecting and/or maintaining the beneficial commensal flora on the skin and/or mucosa.

4. The method as claimed in claim 3 for preventing and/or reducing and/or delaying the unesthetic and/or unpleasant and/or uncomfortable manifestations of the skin and/or mucosa whose beneficial commensal flora is altered, in particular for preventing and/or reducing and/or delaying the secretion of sebum and its unesthetic and/or unpleasant and/or uncomfortable manifestations such as for preventing and/or reducing and/or delaying the formation of blackheads and/or comedogenesis and/or the shiny appearance of the skin, and/or for maintaining and/or improving the homogeneity of the complexion of the skin and/or the mucosa and/or reducing redness, and/or for preventing and/or treating the feeling of warmth and/or heat of the skin and/or the mucosa, and/or for preventing and/or reducing head hair loss and/or body hair loss and/or for preventing and/or reducing dandruff.

5. The method as claimed in claim 3, characterized in that the beneficial commensal flora is a microorganism chosen from the group made up of Staphylococcus epidermidis, Acinetobacter lwoffii and mixtures thereof.

6. The as claimed in claim 1, characterized in that the protein extract of ungerminated, deoiled Moringa oleifera seed is applied topically, to specific parts of the body chosen from the legs, feet, underarms, hands, neck, chest, stomach, arms, thighs, hips, buttocks, waist, crotch, groin, torso, back, labial mucosa, face and/or scalp.

7. The method as claimed in claim 1, characterized in that the protein extract of ungerminated, deoiled Moringa oleifera seed is a water soluble extract.

8. The method as claimed in claim 1, characterized in that the protein extract of ungerminated, deoiled Moringa oleifera seed contains, on the basis of the dry extract, a protein content, particularly of native proteins, comprised between 0.01 and 100% by weight.

9. The method as claimed in claim 1, characterized in that the protein extract of ungerminated, deoiled Moringa oleifera seed does not contain alkaloids, Pterygospermin, isothiocyanates or kaempferol.

10. The method as claimed in claim 1, characterized in that the protein extract of ungerminated, deoiled Moringa oleifera seed is an extract of hulled seeds.

11. The method as claimed in claim 1, characterized in that the protein extract of ungerminated, deoiled Moringa oleifera seed is found in the form of a cosmetic ingredient intended to be incorporated in a cosmetic composition, and also comprising an appropriate cosmetic vehicle.

12. The method as claimed in claim 11, characterized in that the protein extract of ungerminated, deoiled Moringa oleifera seed is present in the cosmetic ingredient in the liquid form in a content comprised between 0.01 and 10% by weight of dry matter relative to the total weight of the ingredient, or in the solid form in a content comprised between 10 and 60% by total weight of the ingredient.

13. The method as claimed in claim 1, characterized in that the protein extract of ungerminated, deoiled Moringa oleifera seed or the cosmetic ingredient comprising same is found in the form of a cosmetic composition intended for topical administration also comprising an appropriate cosmetic vehicle.

14. The method as claimed in claim 13, characterized in that the protein extract of ungerminated, deoiled Moringa oleifera seed is present in the cosmetic composition in a content comprised between 0.0001 and 20% by weight of dry matter relative to the total weight of the composition.

15. The method as claimed in claim 13, characterized in that the composition is in the form of a serum, a lotion, a cream, a milk, an ointment, a paste, a foam, an emulsion, a hydrogel, a shower gel, an aerosol, a mask, a stick, a patch, or face powders.

16. The method as claimed in claim 1, characterized in that the protein extract of ungerminated, deoiled Moringa oleifera seed or the cosmetic ingredient comprising same is found in the form of a cosmetic composition intended for oral administration, also comprising an appropriate cosmetic vehicle.

17. A protein extract of ungerminated, deoiled Moringa oleifera seed, for its use in the treatment of reactive, hyperreactive, intolerant and/or irritated skin and/or mucosa and/or in the treatment and/or prevention and/or reduction of the occurrence of pathologies related to skin and/or mucosa that are sensitive, sensitized, reactive, fragile, weakened, intolerant, hyperreactive and/or irritated, such as contact urticaria, irritative or allergic contact dermatitis, eczema, psoriasis, seborrheic or atopic dermatitis, and/or in the treatment and/or prevention of inflammation and/or irritation, particularly caused by Staphylococcus aureus, and/or in the treatment and/or prevention of erythema, particularly infant diaper rash and/or in the treatment of gingivitis.

18. The protein extract of ungerminated, deoiled Moringa oleifera seed, for its use as claimed in claim 17 and also for its use in the treatment and/or prevention and/or reduction of the occurrence of pathologies related to an alteration of the cutaneous and/or mucosal beneficial commensal flora, advantageously involving a decrease of the content of beneficial commensal cutaneous and/or mucosal microorganisms and/or an increase in the content of pathogenic microorganisms, preferably in pathogenic bacteria, in particular Staphylococcus aureus and/or Propionibacterium acnes, advantageously pathologies chosen from the group consisting of infections, especially bacterial, of the skin and/or mucosa, ulcers, herpes, boils, folliculitis, abscesses, sycosis, impetigo, ecthyma erysipelas, acne, fungal infections such as candidiasis or dermatophytosis, such as ringworm and/or scabies and/or in the treatment and/or prevention of wound infections, and/or in the prevention of pigment spots and/or acne scars.

19. The protein extract of ungerminated, deoiled Moringa oleifera seed, for its use as claimed in claim 17, characterized in that the protein extract of ungerminated, deoiled Moringa oleifera seed is a water soluble extract.

20. The protein extract of ungerminated, deoiled Moringa oleifera seed, for its use as claimed in claim 17, characterized in that the protein extract of ungerminated, deoiled Moringa oleifera seed is found in the form of a pharmaceutical ingredient or a pharmaceutical composition also comprising an appropriate pharmaceutical vehicle.
